(19) United States
(12) Patent Application Publication
(10) Pub. No.: US 2004/0162549 A1
(43) Pub. Date: Aug. 19, 2004

(54) Method and apparatus for performing optical dermatology

(75) Inventor: Gregory B. Altshuler, Wilmington, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Palomar Medical Technologies, Inc., Burlington, MA

(21) Appl. No.: 10/706,721

(22) Filed: Nov. 12, 2003

Related U.S. Application Data
(60) Provisional application No. 60/425,983, filed on Nov. 12, 2002.

(51) Int. Cl. A61B 18/20

(52) U.S. Cl. 606/9

(57) ABSTRACT

The invention provides methods and systems for performing optical dermatology employing a plurality of optical radiation sources that can be selectively operated in a predetermined pattern or sequence. An optical dermatology apparatus is disclosed having a mount adapted for positioning in proximity of an area of a patient's skin, one or more radiation sources disposed in the mount for irradiating at least a portion of the area of the patient's skin, and a control circuitry electrically coupled to the radiation sources for actuating a selected pattern or sequence of the radiation sources for performing a treatment protocol. The mount can be shaped to substantially conform to a patient's body part, such as a face mask. The invention also discloses using one or more sensors disposed in the mount such that the patient's skin can be monitored. A computer in communication with the applicator can receive data from the sensors and transmit control signals to the control circuitry based on analysis of the data.

### PRIORITY

This application claims priority to U.S. provisional application no. 60/425,983 filed Nov. 12, 2002.

### FIELD OF THE INVENTION

This invention relates to methods and apparatus for performing optical dermatology, including cosmetology, and more particularly to such methods and apparatus employing a plurality of optical radiation sources operated in a predetermined pattern or sequence.

### BACKGROUND

Optical radiation has been utilized for many years in both medical and non-medical settings for various therapeutic and cosmetic dermatology treatments and procedures. Such treatments include, but are by no means limited to, management of hair growth (removal of unwanted hair, stimulating or slowing hair growth, etc.), treatment of ingrown hairs, also known as Pseudofolliculitis Barbae (PFB) and “razor bumps,” improvement of skin quality (for example, correcting skin pigmentation problems, skin texture, wrinkles and elasticity), treating vascular problems (for example, spider veins, rosasea, varicose veins, port wine stains and other vascular lesions), treatment of psoriasis, pigmented lesions, photodamaged skin, scars, stretch marks, acne, tattoo removal and the like. Both coherent (for example, laser) and incoherent (for example, various lamps, light emitting diode) light sources have been used in performing such treatments and procedures.

Most of these treatments have heretofore been performed in a medical environment, and even where such procedures are currently performed in a spa or salon, medical personnel are normally involved. Reasons for operating in such environments include the fact that the equipment required to perform such procedures is currently relatively large and expensive, and, normally involving an optical radiation source producing relatively high energy, presents significant risks both to the operator and the subject if the procedure is not performed properly. In particular, radiation from the source can cause damage to the eyes or other body parts of the subject on which treatment is not intended or to the operator and overexposure of a portion of the subject's skin can result in pain and thermal damage. Conversely, inadequate exposure to the radiation can prevent desired therapeutic effects from being achieved.

Existing devices, regardless of the light source employed, have been of four general types. Most of these devices have utilized a head through which radiation from a pulsed light source is applied to a treatment region. Generally after each pulse, but sometimes after more than one pulse, the operator may reposition the head to treat a new region in a larger treatment area. The head may either be in contact with or slightly spaced from the subject's skin during the treatment.

The second procedure is to apply radiation from a continuous wave (CW) source through the head and to move the head over the area to be treated at a selected rate to effect the treatment. The head may be moved to perform multiple passes over a particular treatment region.

The third technique is to mount the head, which may pass radiation from either a pulsed or CW source, in a scanner device which device is mounted over the treatment area, the scanner device moving the head in a predetermined stepped or continuous movement pattern over the treatment area to effect the desired treatment.

The fourth type utilizes a head with large emitting area for treatment of a large part of body, for example a tanning chamber or UVB lamps for psoriasis treatment. Such devices can for example comprise several lamps or LED's, which radiate simultaneously in CW or pulse modes.

As indicated earlier, all of these techniques have disadvantages which prevent their use by other than highly trained medical and other personnel. First, the treatments are highly dependent on the skill of the operator. In particular, both the placement of the apparatus for treatment and the dwell time of the apparatus on each treatment region are normally controlled by the operator. Thus, a skilled operator is required both for efficacy of the treatment and safety. A skilled operator is also required to prevent damage to a subject's eyes or to other areas not intended for treatment. While the scanner devices or head with large emitting area can somewhat reduce these problems, the high cost and complexity of these devices limit their use outside of institutional settings.

A need therefore exists for a relatively simple, safe and inexpensive method and apparatus for performing medical and cosmetic dermatology procedures which is simple enough and foolproof enough so as to be operated by untrained or minimally trained personnel, or even by the person on whom the procedure is to be performed, while also being sufficiently foolproof so that there is no danger of injury to the subject's eyes or other unintended parts of the subject's body or overexposure of a treatment region of the subject's body, while still assuring that an effective treatment is performed.

### SUMMARY OF THE INVENTION

The invention provides methods and systems for performing optical dermatology employing a plurality of optical radiation sources that can be selectively operated in a predetermined pattern or sequence. An optical dermatology apparatus is disclosed having a mount adapted for positioning in proximity of an area of a patient's skin, one or more radiation sources disposed in the mount for irradiating at least a portion of the area of the patient's skin, and a control circuitry electrically coupled to the radiation sources for actuating a selected pattern or sequence of the radiation sources for performing a treatment protocol. The mount can be shaped to substantially conform to a patient's body part, such as a face mask. The invention also discloses using one or more sensors disposed in the mount such that the patient's skin can be monitored. A computer in communication with the applicator can receive data from the sensors and transmit control signals to the control circuitry based on analysis of the data.

In one aspect the invention discloses an optical dermatology apparatus having a plurality of optical radiation sources, a mount in which the sources are positioned at selected locations, and controls for operating the sources in a selected pattern. The mount can be adapted for positioning adjacent or in proximity to a treatment region of a subject's body. The apparatus can be controlled such that a selected percentage of the sources can be operated at a time. The sources can be individually operated in the selected pattern or selected sequence. The controls include one or more switching elements, which selectively connect the power supply to the sources in the selected pattern or sequence.

The mount can include a component which protects a selected portion of the subject's body by preventing application of light from the sources to the selected portion. For example, the treatment region can be the subject's face and wherein the selected portion is the subject's eyes. In one embodiment, the apparatus can include an interlock which operates in conjunction with the controls to disable operation of the sources unless the component is properly positioned to protect the selected portion.

The mount can also include a component which permits the mount to be fitted to the treatment region with substantially uniform spacing between each the sources and the treatment region. The mount can include an optically transparent component between the sources and the treatment regions. The transparent component can also include an optical filtering component, which can be a protective component for the subject The mount can also include a circuit board, such that the sources can be mounted to the board. The mount can be adapted to fit to all or part of a subject's face. The treatment region can be any portion of the subject's skin, for example, the subject's face, arm, thigh, leg, arm, hand, neck, hairline, underarms, back, crouch area, bikini line, buttocks, breast, or stomach. The mount can be adapted to be fitted to the treatment region.

The apparatus can include a surface for reflecting radiation from the sources to the treatment regions. There can be a reflecting surface for each of the sources. The sources can be U-shaped lamps. The sources can be solid state light emitters. A lens array can be included in the apparatus for directing light from the light emitters to the treatment region.

In another embodiment, the apparatus can further include a diagnostic tool mounted in the mount. The controls can operate in response to the diagnostic tool to control operations of the sources. The apparatus can include sensors for detecting proper positioning of the mount relative to a protected portion; whereby the controls operate in response to the sensors to operate the sources only when the mount is properly positioned.

In another aspect, the invention provides a method of performing optical dermatology by operating at least some of a plurality of optical radiation sources mounted adjacent a treatment region of a subject in a selected pattern or sequence. A small or selected percentage of the sources can be operated at a time. For example, specific subsets of the sources can be operated simultaneously. The specific subsets can be operated in a selected sequence. This can be used, for example, to irradiate specific regions of the skin while other regions of the skin are not irradiated. The sequence can be such that a specific region of the skin can be irradiated multiple times interspersed with periods of non-irradiation. The sources can also be individually operated in the selected sequence.

The method can further include protecting a selected portion of the treatment region by preventing application of light from the sources to the selected portion. For example, the treatment region can be the subject's face and wherein the selected portions in the subject's eyes. The protecting step can include detecting that the selected portion is properly protected, and enabling operation of the sources in response to the detection.

The method can include setting a mount for the sources to the treatment region so as to provide substantially uniform spacing between each the source and the treatment region. The treatment region can be the subject's lower face, entire face, arm, thigh, leg, arm, hand, neck, hairline, underarms, back, crouch area, bikini line, buttocks, breast, or stomach.

The method can further include detecting a selected condition of the treatment region. The sources can be operated in response to the detection. The method can include sensing the proper positioning of a mount for the radiation sources relative to the treatment position; and operating the sources only when the sensing step indicates that the mount is properly positioned.

In another embodiment, the invention provides an optical dermatology apparatus having a mount adapted for positioning in proximity of an area of a patient's skin, one or more radiation sources disposed in the mount for irradiating at least a portion of the area of the patient's skin, and a control circuitry electrically coupled to the radiation sources for actuating a selected pattern of the radiation sources for performing a treatment protocol. The mount can be shaped so as to substantially conform to a contour of a patient's body part. The treatment protocol can consist of selecting wavelength(s) appropriate for a dermatological condition. The control circuitry can be programmed to selectively treat a portion of the treatment area. The control circuitry controls can activate the selected pattern of the radiation sources in a selected sequence.

In one embodiment, the mount is disposable. Alternatively, the apparatus can further include an optically transparent sheath adapted to couple to the mount, wherein the sheath conforms to the treatment area. The sheath can be user-replaceable.

In yet another embodiment, the invention discloses an optical dermatology system having an applicator which comprises a mount for positioning in proximity of an area of a patient's skin, a plurality of radiation sources disposed in the mount, one or more sensors disposed in the mount for collecting any of diagnostics or monitoring data associated with at least a portion of the area of the patient's skin, a control circuitry electrically coupled to the radiation sources for selective actuation thereof, and a computer in communication with the applicator The computer can receive data from the sensors and transmitting control signals to the control circuitry based on analysis of the data. The control signals can cause actuation of at least selected ones of the radiation sources in a selected sequence. The computer can communicate with the applicator via a wireless communications link.

### BRIEF DESCRIPTION OF DRAWINGS

Other objects, features and advantages of the invention will be apparent from the following more particular description of various embodiments of the invention as illustrated in the accompanying drawings.

FIG. 1 is a side view of an apparatus in accordance with the teachings of this invention adapted for treating the lower part of the face;

FIG. 2 is a side view of a device of the type shown in FIG. 1 modified for treatment of a full face;

FIG. 3 is a side view of a device in accordance with the teachings of this invention adapted for treatment of the neck, for example, trimming or removing hair from a subject's neck;

FIG. 4 is a front view of a device in accordance with the teachings of this invention for treatment of a subject's underarms;

FIG. 5 is a side sectional view of an applicator suitable, in accordance with the teachings of this invention, for treating hand(s);

FIG. 6 is a schematic diagram of a device suitable for treating cellulite on a patient's buttocks;

FIG. 7 is a schematic diagram of a photofacial treatment device that is combined with a cordless base unit;

FIG. 8 is a partially cut away side view of a face mask applicator suitable for use in practicing the teachings of this invention;

FIG. 9 is a longitudinal cross-section view of a portion of an applicator suitable for practicing the teachings of the invention;

FIG. 10 is a longitudinal cross-section view of a portion of an applicator for an alternative embodiment of the invention;

FIG. 11 is a longitudinal cross-section view of a portion of an applicator for still another embodiment of the invention;

FIG. 12 is an electrical schematic diagram of a device suitable for practicing the teachings of the invention;

FIG. 13 is a schematic diagram of an optical dermatology system according to one embodiment of the invention that includes an applicator, a diagnostics sensor having a CCD camera, and a treatment module; and

FIG. 14 is a schematic diagram of a photofacial treatment device with an integrated design of sensors and radiation sources.

### DETAILED DESCRIPTION

In accordance with the teachings of this invention, a plurality of optical radiation sources are fixedly positioned or mounted in a suitable applicator or mount. The optical radiation sources may be lasers, for example diode lasers, or other coherent light source or may be some form of lamp or other non-coherent light source, for example an arc (Xe, Kr), Ar, Ne, Hg, metal halide, halogen, etc. lamp or light emitting diode (LED). The light source utilized will depend on many factors, including price target, the treatment to be performed and the treatment protocol. The radiation sources are controlled from a programmable power source so as to provide an irradiation pattern. The term “pattern,” as used herein, is intended to encompass spatial patterns, e.g., illumination by all or a subset of the sources for a period of time, as well as temporal patterns, e.g., sequential illumination of a treatment region by certain sources. The irradiation pattern may be fixed for a particular apparatus, may be programmable within certain safe limits by the user or, as will be described in greater detail later, may be automatically determined by the apparatus in response to a diagnostic operation performed by suitable components which form part of the apparatus.

Safety is enhanced by providing interlocks which prevent operation of the apparatus unless the apparatus is properly positioned on a treatment area and/or protective members are suitably positioned to assure that radiation is not applied to the eyes or other unintended areas of the body. Thus, since the operator cannot cause radiation to be applied unless unintended areas of the body are protected and the operator can cause neither a sufficient dose of radiation to be applied to cause injury to a subject's skin nor an insufficient amount of radiation to be applied so that the desired treatment is not performed, the apparatus can be safely and efficaciously used by unskilled operators, for example beauticians, barbers and the subject herself.

Further, since the radiation sources can each be relatively inexpensive, the need for 2-50 or more radiation sources for a given device still permits the devices to be relatively inexpensive. The electrical controls required for operating the device are also relatively inexpensive, so that the total cost of the device should be low enough to facilitate use in barber shops, beauty salons and similar businesses or even home use. In some embodiments, cooling is not required. The power source required to operate only a single source, or at most a relatively small number of the sources at a time, can also be relatively inexpensive. Since each source generates a relatively small amount of radiation on a relatively small area of the subject's body, cooling should not be required to prevent overheating and damage to the radiation source of the applicator or to the subject's skin. In other embodiments, cooling may be desirable and can be accomplished by various methods known in the art.

Examples of applications of aspects of the invention include, but are not limited to, skin texture improvement, scar removal or healing, wrinkle removal, skin tightening, skin elasticity improvement, skin thickening, skin rejuvenation, cellulite treatment/fat reduction, vascular and lymph regeneration, subcutaneous collagen structure improvement, acne treatment, psoriasis treatment, fat reduction, hair growth stimulation, treatment of alopecia, treatment of lentigo senile, treatment of striae, pain relief, wound healing, healing of epidermis and dermatitis, treatment of eczema, treatment of decubitus ulcer, healing of haematoma, treatment after skin resurfacing, odor reduction, muscles contraction relaxation, reduction of gum inflammation, reduction of pulpitis, treatment of herpes, treatment of alveolities, aphtae and hyperemia, reduction of oedema, drum healing, treatment of tinnitus, reduction of microscars and polyposis, treatment of adnexitis, bartholinitis, cervicitis, epiziotomy, HPV, menorrhagia, and parametritis, and vulvitus. Non-limiting wavelength ranges that can be used to treat a variety of diseases and cosmetic conditions can be found in Table 1.

Treatment with the apparatus proposed in present invention can be combined with other treatments. For certain applications, such as acne, compression of the skin can lead to better penetration of light to the sebaceous follicle including the gland. The optical treatment can be combined with cleaning of comedo and sebaceous follicle opening. The optical treatment can also be used in combination with anti-bacterial and or anti-inflammatory lotions, which can be applied before and/or after optical treatment. The apparatus of the present invention can also be used in combination with topical substances, such as a light activated lotion, for example, a lotion with a photosensitizer or photosensitizer production compound such as 5-aminolevulinic acid (ALA). Additionally, a lotion can be applied that contains absorption compounds, such as carbon, melanin, or a dye that increases light absorption resulting in better heating effects. The concentration of photosensitizer should be below a threshold of side effects from sun and other lightening systems, but above a threshold of photochemical effect on hair follicles, sebaceous glands or sebaceous follicles from a light emitting applicator. As a result, this treatment can be effective on hair growth, acne, skin oiliness, skin tone and skin texture.

TABLE 1
Examples of wavelength ranges useful for the treatment of
specific diseases and cosmetic conditions.

| | |
|---|---|
| Treatment Condition or Application | Wavelength, nm |

Dermatology/Cosmetology

| | |
|---|---|
| Acne | 290-700, 900-1850, |
| | 390-450 and 600-700 |
| ALA lotion with PDT effect on skin | 290-700 |
| condition including anti cancer effect | |
| Alopecia | 620-680 and 760-880 |
| Anti-aging | 400-2700 |
| Blood, lymph, immune system | 290-1350 |
| Cellulite | 600-1350; 760-880 |
| Color lotion delivery into the skin | Spectrum of absorption of |
| | color center and 1200-20000 |
| Deep vascular | 500-1300 |
| Deep wrinkle, elasticity | 500-1350 |
| Direct singlet oxygen generation | 1260-1280 |
| Hair growth control | 400-1350 |
| Hair growth stimulation | 600-700 and 760-880 |
| Lentigo senile | 600-700 |
| Lotion delivery into the skin | 1200-20000 |
| Lotion with PDT effect on skin condition | Spectrum of absorption |
| including anti cancer effect | of photo sensitizer |
| Muscular, joint treatment | 600-1350 |
| Odor | 290-1350 |
| Oiliness | 290-700, 900-1850 |
| Pain relief | 500-1350 |
| PFB | 300-400, 450-1200 |
| Pigmented lesion, de pigmentation | 290-1300 |
| Psoriasis | 290-700; 600-700 |
| Scars | 380-420, 620-680 |
| | and 760-830 nm |
| | (depending on scar nature) |
| Skin cleaning | 290-700 |
| Skin lifting | 600-1350 |
| Skin rejuvenation | 600-700 and 760-880 nm |
| Skin texture, stretch mark, scar, porous | 290-2700 |
| Striae | 760-880 nm |
| Superficial vascular | 290-600 |
| | 1300-2700 |
| Wrinkles | 620-680 and 760-880 nm |

Dentistry

| | |
|---|---|
| Gingivitis | 380-450 and 600-700 nm |
| Gum inflammation | 380-450 and 600-700 nm |

Other

| | |
|---|---|
| Burns | 760-880 nm |
| Pain relief | 760-880 nm |
| Wound healing | 380-1250 nm |
| | (depending on wound nature) |

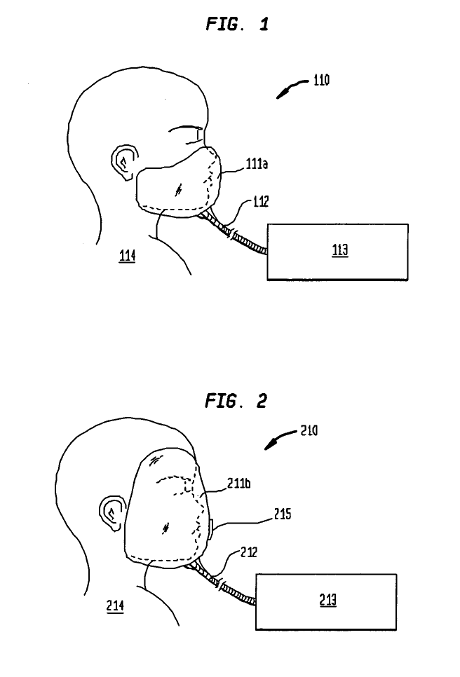

The figures illustrate various embodiments of the invention adapted for treating various parts of the body. In particular, FIG. 1 shows an apparatus 110 suitable for treating the lower portion of the face of a subject 114. The apparatus might, for example, be utilized for the removal of facial hair on a man or woman. By using relatively low power radiation source(s), the apparatus could be utilized for temporary facial hair removal prevention of razor bumps (PFB) and/or to impede or delay hair growth so that the subject 114 may need to shave only once a week or once a month, rather than every day. Apparatus 110 consists of an applicator or mount 111a, which, for this embodiment, is in the form of a half-mask that is fitted to the subject's face in a manner to be described in greater detail below. The apparatus also includes a control box 113 that may include a power supply and suitable control electronics. The power supply may be adapted to be plugged into an ordinary electrical outlet or other suitable power supply or may be operated from a battery or other suitable power source which could also be contained in the box 113. Box 113 is connected to applicator 111a through an umbilical 112 through which both power and control signals may pass. As will be discussed later, diagnostic and/or feedback information may also pass from the applicator to the controls of box 113. Further, while the power supply and controls of box 113 are connected to applicator 111 through an umbilical 112 for illustrative embodiments, this is not a limitation on the invention and, particularly where the power supply and controls are small enough, they may be connected directly to an applicator 111, integrated with the applicator or both may be connected to a common mounting element containing connective circuitry. Other arrangements may also be possible. However, for currently available components, the configuration shown in FIG. 1 is considered preferable.

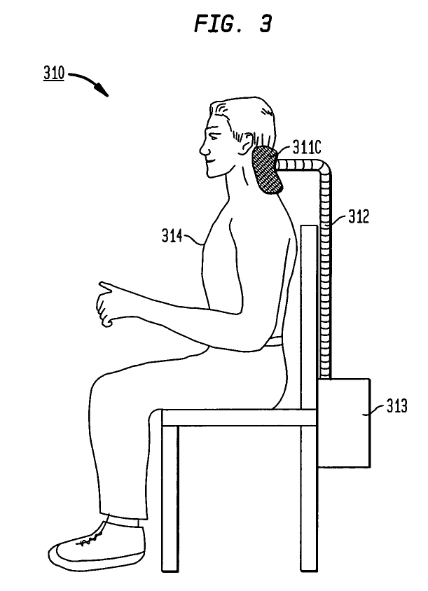

FIG. 2 shows an embodiment of the invention that differs from that of FIG. 1 only in that the applicator 211b for this embodiment is adapted to fit over the subject's 214 entire face, rather than just the lower part of the subject's face. This embodiment might, for example, be utilized for wrinkle removal or other treatments involving anti-aging treatment, improvement of, texture, porous, wrinkle, scar, vascularity, hyper or hypopigmentation, skin tone, redness, facial skin quality, sagging, skin lifting, for facial hair removal, for treating facial acne in different face including its prevention or reduction of oiliest and for other suitable applications. A suitable mechanism 215, such as a breathing hole for example, can be included to permit the subject to breathe through applicator 211b. Further, as discussed in greater detail later, a mechanism would also be provided to protect the subject's eyes and prevent radiation from being applied thereto and to inhibit operation of the apparatus 210 unless such protective gear was properly in place. Apparatus 210 includes a control box 213 that may include a power supply and suitable control electronics. Box 213 is connected to applicator 211b through an umbilical 212 through which both power and control signals may pass. Components are substantially as described for FIG. 1.

FIG. 3 illustrates still another embodiment of the invention wherein the apparatus 310 has an applicator 311c that is adapted to be fitted to the back of the subject's neck. For this embodiment, box 313 is also shown as attached to the chair in which subject 314 is sitting, rather than resting on an adjacent table, but the position of the box 313 is a matter of design choice. Applicator 311c can, for example, be used to remove hair growing on the subject's neck and/or to trim the subject's hair. Controls 313 can be programmable to control the length and shape of the hairline of subject 314. Box 313 is connected to applicator 311c through an umbilical 312. Components function substantially as for the prior embodiments.

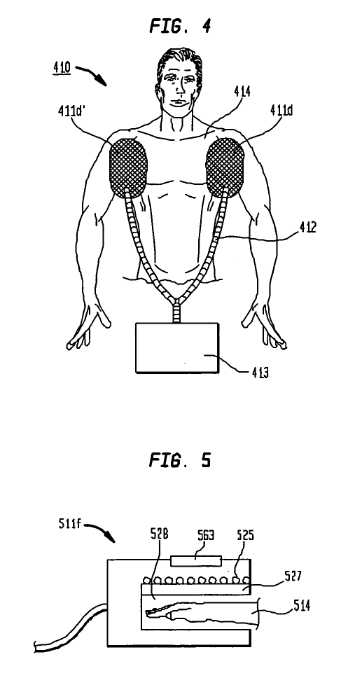

FIG. 4 illustrates an embodiment of the invention wherein the apparatus 410 has applicators 411d that are adapted to be fitted to the subject's 414 underarms to, for example, remove unwanted hair therefrom, odor, antiperspirant treatment. Control box 413 is connected to the applicator(s) 411d through an umbilical 412.

In another embodiment, an applicator can be adapted to be fitted in the crotch area of the subject to, for example, remove pubic hair, and in particular hair which might extend from the bikini line. The applicator can be hand held or self-supporting. For example, the applicator can be supported on a curved wire or pole attached at each end to a foot bar. In another embodiment, the applicator can be attached to an adjustable pole, so that the subject can position the applicator at the correct height and location for the desired hair removal. Other techniques for supporting applicator are also within the contemplation of the invention. This embodiment would also have controls connected to the applicator either through an umbilical cord, wireless connection, or other suitable connection mechanism.

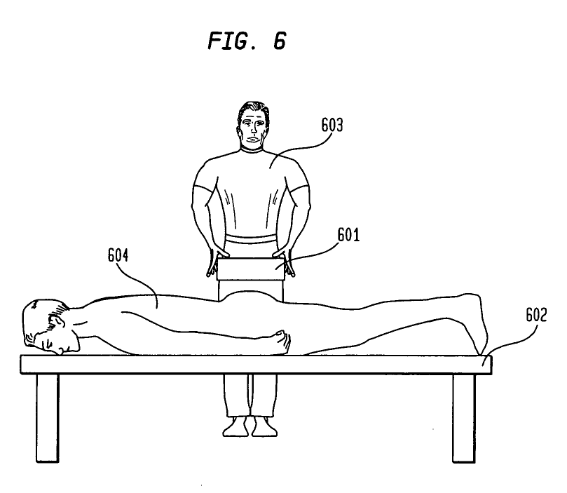

FIG. 5 shows an applicator 511f for treating the hand of subject 514 to, for example, remove age spots or other pigmented lesions therefrom or to otherwise improve the skin quality of the hand by improving elasticity and/or removing wrinkles therefrom. Umbilical 512 for this embodiment would also be connected to suitable controls (not shown). Details for this embodiment of the invention will be described later.

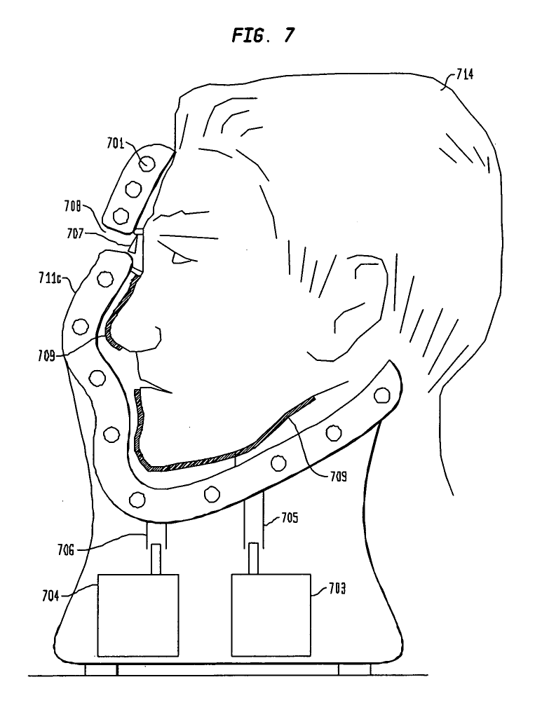

FIG. 6 shows another embodiment of the present invention in which the applicator 601 can be held by an operator 603 for example for treatment cellulite on a buttocks of a patient 604 lying on a table 602. The applicator 601 can be adapted to conform to the patient's buttocks. The applicator 601 can also be used to remove hair, tighten skin, remove acne or any other of the applications detailed in Table 1.

FIG. 7 shows a photofacial treatment device which is combined with a cordless base unit, i.e., without umbilical. Light sources 701 can be mounted in the body of the face mask 711c which is connected with electronics 703. Air cooling devices 704 can be connected through connectors 706 or 705. In one embodiment, the air cooling devices 704 can house a phase change material, i.e., liquid nitrogen. Alternatively, the air cooling device can be an air pump, airflow device or fan that can be used for skin cooling and comfort. The mask can have opening 708 for eyes so that the subject 714 can see. The mask can also have adjustable, see-through eye shields 707. In one embodiment, the photofacial treatment device can further include an optically transparent sheath 709 adapted to couple to the mask 711c, wherein the sheath 709 can conform to the subject's face. The sheath 709 can be user-replaceable.

All masks and/or skin contacting elements of the applicators of the present invention can be disposable. For example, several masks with different spectra can be supplied with one unit. Each mask can be optimized for different treatments, such as, for example, acne, hair, or skin rejuvenation.

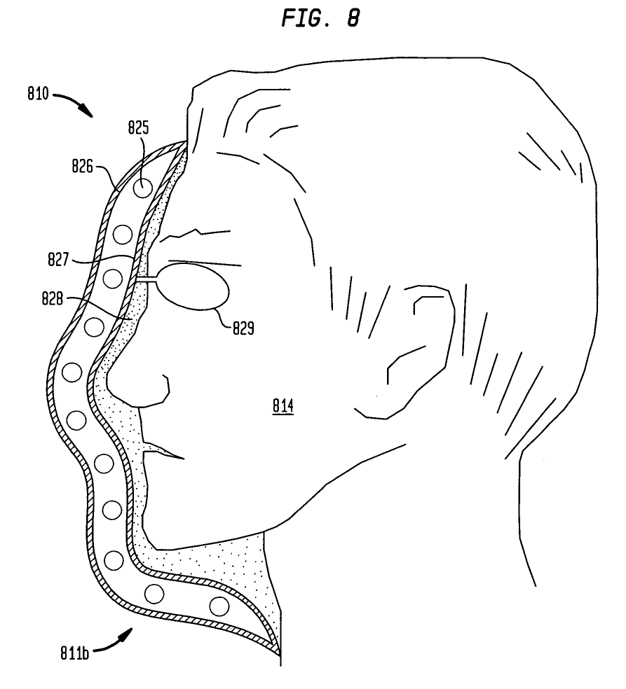

FIG. 8 is a more detailed cross-sectional view of an applicator 811b or mask such as the applicator 211b shown in FIG. 2. For this embodiment, a plurality of light sources 825 are provided which are arranged in an appropriate predetermined pattern. While the sources, the nature of which has been previously discussed, may be spaced as shown in FIG. 8, they may also be mounted more closely together or further apart to form a matrix of small light sources. Further, while one column of sources 825 is shown in FIG. 8, multiple columns of lamps can normally be provided around the applicator. Alternatively, the pattern in which the sources are mounted does not require that they be mounted in columns. Sources 825 can, for example, be embedded in a suitable light-transmitting material or can otherwise be suitably mounted in the applicator. The applicator has an outer wall 826 and an inner wall 827. The outer wall 826 forms a protective cover for the applicator. The inner surface of the outer wall or cover 826 is preferably reflective or is coated with a specula or diffuse reflective material so as to reflect light impinging thereon toward subject 814. This improves the efficiency of the apparatus, permitting substantially all of the light from sources 825 to be directed to subject 814. Where sources 825 are of a type which emit light only in the direction of the subject, it may not be required for rear wall 826 to be reflective. However, the reflector may also be utilized to retro-reflect light emitted from the subject's skin as a result, for example, of scattering, this retro-reflection, or photon recycling, thereby further enhancing the efficiency of the apparatus 810. While rear wall 826 may be solid, it is preferable that it be flexible to permit it to conform to the shape of the subject's face or to the shape of some other part of the subject's body on which treatment is to be performed. In another embodiment, the rear wall can have outlets to allow air to pass through. In yet another embodiment, the mask can be solid and adapted for coupling to the patient's face. For example, an adjustable head rest and/or chin rest can be attached to the mask allowing it to be positioned at the optimum distance from the patient's face.

Front wall 827 is formed of an optically transparent material, at least for the portion of the optical spectrum to be utilized for the desired procedure. Wall 827 may be formed of a material or may be coated with one or more layers of material, may have a layer of material mounted adjacent thereto or may otherwise be provided with a filtering capability to assure that only desired wavelengths of light pass therethrough to subject 814 to effect the desired treatment. Filtering out undesired wavelengths of light where a broadband optical radiation source is utilized is one way in which the skin of subject 814 is protected against thermal injury. Wall 827 can also be formed, coated or otherwise adapted as light scattering wall to increase the uniformity of light radiation on the skin. Where the radiation source 825 is a lamp or other source which may explode or shatter, wall 827 should be formed of a hard material so as to protect subject 814 from injury in the event of any such explosion.

The gap between front wall 827 and the subject's skin should be maintained as small as possible so that radiation sources 825 are as close to the subject's skin as possible; however, it is preferable that the entire applicator be designed so as to maintain a substantially uniform spacing between radiation sources 825 and the subject's skin. Non-uniformity in this spacing can lead to non-uniformity in treatment and makes control of the treatment far more difficult. Gap 828 can be filled with air or by a flowing gas to cool and protect the subject's skin. However, air in gap 828 results in an optical mismatch at the gap interfaces and is therefore not preferred. Further, if the radiation sources 825 are properly selected and operated, cooling of the subject's skin should not be required.

It is therefore preferable that gap 828 be filled with a viscous gel or lotion or be filled by an elastic mask made of an optical resin (silicon) or similar material. This mask can be made as a double replica of the area to be treated, can be synthesized from a 3-D photograph (digital or analog) or can be of a material which is soft enough to mold to the subject's face when applied to the face and which then sets to conform to the subject's face. The material used should provide a good optical match with the subject's face/skin to minimize optical discontinuities. Such a fill can be more comfortable for the subject, can assure proper positioning of the applicator on the subject's face and, by permitting tight control of the spacing between the radiation source(s) and the subject's skin, provide more efficient and safer operation. While not shown in the figure, a facility would be provided to permit subject 814 to breathe either through his nose or mouth, for example a tube passing through and being sealed in applicator 811b which goes into the subject's mouth and through which the subject can breathe.

A key safety feature of applicator 811b is protective mechanism 829 which, for the embodiment shown, includes a pair of eye shields mounted to wall 827 and positioned to fit over the subject's eyes when applicator 811b is properly positioned on the subject's face. Eye protection mechanism 829 is optically opaque so that optical radiation cannot pass therethrough to the subject's eyes. Eye protectors 829 are preferably spring loaded and a suitable sensor is provided to prevent operation of radiation sources 825 until eye protectors 829 have moved a predetermined amount against their spring load, thereby assuring that the eye shades are properly positioned over the subject's eyes. Sensors may be provided at other places on the applicator to assure proper positioning of the applicator on the subject's face, with interlocks being provided to prevent operation of radiation sources 825 until all sensors confirm proper positioning of the applicator. With these safety features in place, danger to the subject as a result of improper operation of the apparatus is substantially eliminated.

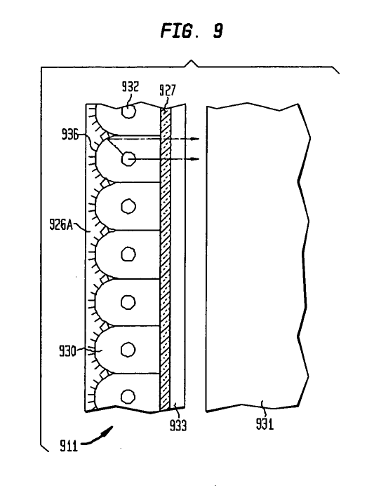

FIG. 9 illustrates in greater detail a possible configuration for an applicator 911 utilizing a lamp 932 as the radiation source. For this embodiment, radiation sources 925 are assumed to be lamps 932, for example, halogen lamps, arc lamps, Xe, Kr, Ar, Ne, Hg, etc. lamps. Each lamp is mounted in its own reflector 936 formed in a rear wall 926a. The gap 930 between each lamp 932 and its reflector 936 may be filled with a gas or other suitable material to provide a good optical match, for example, a condensed medium, so as to enhance optical performance. Light from the lamps and light from reflectors 936 is passed through a front plate or screen 927 that can include filtering as previously discussed and may also be passed through an additional filter 933 to eliminate unwanted wavelengths. This filter can, for example, be a polymer film doped with dye. Layer 927 and/or 933, either instead of or in addition to being doped with a dye, can be coated with a multi-layer interference filter. The filter can be, for example, a fluorescence filter and can be designed to minimize heating and maximize energy. Light passing through filters 927 and/or 933 is applied to skin 931 of subject 914. Additional filtering may be provided by the lamp covers or balloons by a coating on the balloons, by materials in the gaps between the lamps and reflectors and/or by the reflectors themselves or coatings on the reflectors.

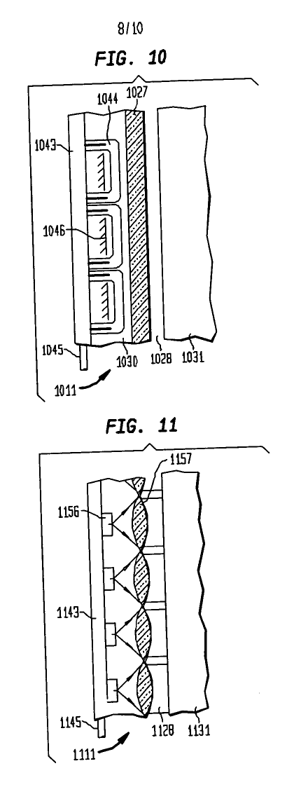

FIG. 10 shows still another embodiment of the invention wherein the applicator 1011 includes a plurality of U-shaped lamps 1044 mounted in a circuit board 1043 which either forms the rear wall of the applicator or is mounted to such rear wall. The U-shaped lamps may be arranged in a selected pattern on the circuit board. Each lamp 1044 has a mirror 1046 positioned behind it on circuit board 1043, which mirrors perform substantially the same function as the mirrors 936 of FIG. 9. Wiring 1045 for energizing and controlling lamps 1044 passes through circuit board 1043. Front plate 1027 performs the same filtering and other functions as for prior embodiments and gap 1028 between the front plate and skin 1031 of subject 1014 and/or gap 1030 between the lamp and the front plate can be filled as previously discussed to enhance delivery of the optical radiation to skin 1031. Where a circuit board 1043 is employed, it is preferably a flexible circuit board so as to facilitate the fitting of the applicator to the treatment area or is preshaped to facilitate such fitting. Walls 1026 and 1027 and filter 1033 are, to the extent used for a particular embodiment, also either flexible or preshaped for the same reason. Walls 1026 and 1027 should provide electrical safety.

FIG. 11 shows still another embodiment for an applicator 1111 wherein the radiation sources 1156 are assumed to be diode lasers, LEDs or similar components emitting radiation in only a single direction. Light from sources 1156 is collimated in lenses 1157, which may be separate lenses or phase screen, but are preferably a multi-lens array formed of plastic or other suitable material, for example a fly's eye lens array. Light sources 1156 are typically substantially monochromatic so that filtering of the output from the sources is not required. However, to the extent filtering is required for an embodiment such as that shown in FIG. 11, lenses 1157 may be coated to provide such filtering or a filter plate may be provided to perform this function. Circuit board 1143 can be connected to a power source via an umbilical 1145. Except for the differences indicated above, the embodiment of FIG. 11, and in particular circuit board 1143 and space 1128, are treated in the same manner as for the embodiment of FIG. 10.

While in the discussion so far, it has been assumed that the radiation sources used in each embodiment are the same, this is not a limitation on the invention. For example, coherent and non coherent light sources can be used in the same device for better treatment, an LED and lamp for example being mounted into every cell of an applicator of the type shown in FIGS. 9-11. In other embodiments, all light sources can be packaged into control box, light being delivered to the applicator through optical fibers or other suitable wave guides in, for example, an umbilical. The output end of each wave guide can be mounted in applicator in a suitable manner in place of the corresponding source, for example, in place of source 1156 in FIG. 11. Several different sources can be mounted in same applicators; for example, LEDs can be used for antibacterial treatment of inflammatory acne and lamps can be used for sebaceous gland treatment.

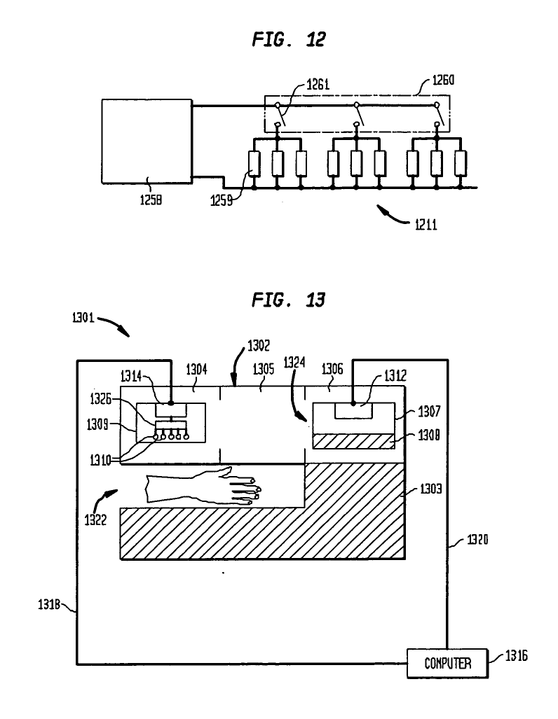

FIG. 12 illustrates an optical schematic for an illustrative embodiment wherein a power supply 1258 energizes a plurality of radiation sources 1259 through a switching array 1260 formed of a plurality of individual switches 1261. While switches 1261 may be mechanical switches, each the switch is preferably an electronic switch. Power supply 1258 may, for example, include a capacitor which charges between pulses and discharges when a switch 1261 is closed to permit power to flow to the radiation sources connected to the closed switch. While three radiation sources are shown connected in parallel or series to each of the switches 1261, this is for purposes of illustration only. Depending on the power supply utilized, the radiation source utilized and other factors, each switch 1261 can energize only a single radiation source or may energize two or more such sources. However, the total number of radiation sources energized off of each switch 1261 should, in accordance with the teachings of this invention, be a very small percentage of the total radiation sources employed; for example, no more than 33%, and preferably no more than 20%. For many applications, the percentage can be under 10%. In this way, the power source utilized may be a relatively small and inexpensive power supply that does not generate substantial heat, and therefore does not require significant, if any, cooling of the control box, the light sources in applicator 1211 or of the subject's skin. Thus, with only a very limited number of radiation sources energized at any given time, the thermal burden on the applicator 1211 is minimized, as heat is both applied to and generated from the subject's skin. This reduced thermal burden should permit the applicator to normally operate without requiring cooling, further reducing the size and cost of apparatus.

Referring again to FIG. 5, applicator 511f of this figure has a slot formed therein in which the subject's hand is positioned. Radiation sources 525 direct light through filter plate 527 and gap 528, which gap may be filled with a suitable gel or other material through which the subject's hand may pass in order to enhance optical match, to the subject's skin. This embodiment also includes a CCD camera, reflectometer or other diagnostic tool which permits the subject's hand to be observed and a determination to be made as to, for example, where on the subject's hand age spots exist that are to be treated. Once such spots are identified, appropriate radiation sources 525 may be operated in a selected sequence to facilitate the treatment/removal of such spots. Diagnostic tool 563 may also be utilized for detecting wrinkles, unwanted hairs or other conditions on which a treatment is to be performed and to provide such information to controls (not pictured) which may then determine appropriate treatment. Diagnostic tool 563 may also be utilized to detect pigmentation of the subject's skin for both this and other embodiments and to utilize this information to select appropriate pulse energy and duration for each radiation source 525 so as to enhance treatment safety while achieving the desired dermatologic result. Having a diagnostic tool such as 563 also permits feedback to be obtained on the treatment, and such feedback can be utilized to control fluence, pulse duration, the lamps to be fired, and other parameters in order to safely achieve a desired result. Safety can also be enhanced by mounting a skin temperature sensor in applicator 511f to monitor skin temperature during treatment and utilizing feedback control to interrupt a light pulse when skin temperature is detected as reaching an exit limited level. The diagnostic system can also measure the end point of treatment.

In some embodiments, diagnostic and/or monitoring sensor(s) disposed in an applicator of the invention, as well as the applicator's control circuitry for selectively actuating the applicator's radiation sources, can communicate with a computer, such as a home personal computer, that is separate from the applicator. The communication between the applicator and the computer can be established by employing any suitable communications protocol, e.g., preferably a wireless communications protocol. The sensor(s) can transmit diagnostics and/or monitoring data regarding a portion of the patient's skin to the computer, which in turn can employ software deployed thereon to analyze the data. The computer can then communicate with the applicator's control circuitry to provide control signals, based on the analysis of the data, thereto.

By way of example, the control signals can direct the control circuitry to actuate a particular pattern of radiation sources, operate selected radiation sources in a sequence appropriate for treatment of an identified skin condition, and/or set radiation fluence of the actuated sources. Alternatively, the computer can analyze, for example, in real time, data received from monitoring sensors and send appropriate control signals to the control circuitry based on the analysis of that data. For example, if the data received from a monitoring sensor indicates that the temperature of a portion of the patient's skin exceeds a pre-defined threshold, the computer can direct the control circuitry to lower the radiation fluence, shut off the applicator, or take any other actions needed to lower to the skin temperature to be in a safe range.

By way of example, FIG. 13 schematically depicts an optical dermatology system 1301 according to one embodiment of the invention that includes an applicator 1302 having a housing 1303 that includes three compartments 1304, 1305, and 1306. A diagnostics sensor 1307 having a CCD camera 1308 is disposed in the compartment 1306, and a treatment module 1309 having a plurality of radiation sources 1310 is disposed in compartment 1304. The diagnostic sensor 1307 and the treatment module 1309 include communications modules 1312 and 1314, respectively, for communicating with a computer 1316, such as a home personal computer, for example, via wireless links 1318 and 1320. The applicator's housing 1303 further includes a slot 1322 in which a patient's body part, e.g., a patient's hand, can be introduced and positioned below the compartment 1305.

Upon positioning of the hand in the housing, the CCD camera can be introduced from the compartment 1306 into the compartment 1305, e.g., via an opening 1324, either manually or under control of the computer 1316 to obtain an image of the patient's hand. The sensor 1307 can transmit the image, via the communications module 1312, to the computer 1316 for analysis. The sensor can be returned to the compartment 1306, and the treatment module can be introduced into the compartment 1305. Upon analysis of the date received from the sensor to identify a particular skin condition, the computer 1316 can transmit control signals to a control circuitry 1326 of the treatment module to actuate selected ones of the radiation sources, or a sequence of radiation sources, to effectuate appropriate treatment protocol. For example, if the analysis of the data indicates the presence of one or more age spots with a pigmentation index of 1380, the computer can cause selective actuation of one or more of the radiation sources at a fluence of about 1310 J/cm² to treat the spots.

Decoupling the computational processing required for data analysis and performing a treatment protocol from the applicator, e.g., utilizing a separate computer for performing such functions, can advantageously lower the complexity and the cost of fabricating the applicator. In addition, it can allow fabricating more compact applicators.

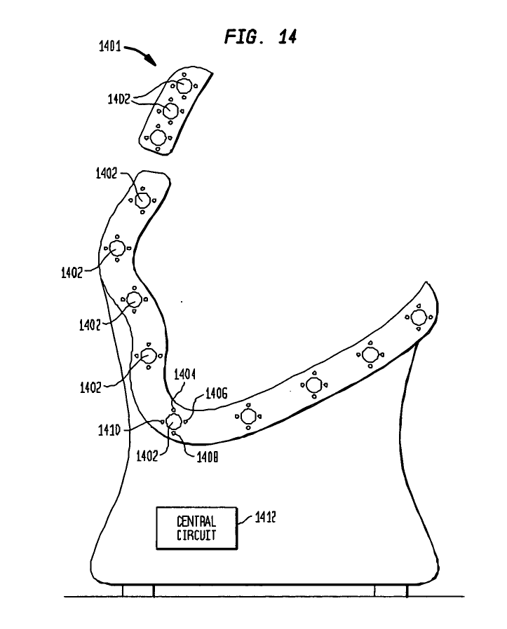

With reference to FIG. 14, in another example, an applicator of the invention, e.g., a mask such as that shown in FIG. 7, can employ an integrated design of sensors and radiation sources. For example, the applicator 1401 can include a plurality of sensors 1402, each of which is surrounded by four radiation sources, e.g. exemplary radiation sources 1404, 1406, 1408, and 1410, that can generate therapeutic radiation of similar to different wavelengths. Each sensor 1402 can be, for example, a thermal sensor that can monitor the temperature of a selected portion of a patient's skin while one or more of the radiation sources associated therewith expose that skin portion to therapeutic radiation. The temperature data can be transmitted to a computer (not shown), e.g., via a wireless connection, to be analyzed and monitored. If the temperature of a skin portion exceeds a pre-defined threshold, the computer can transmit control signals to a control circuitry 1412 of the applicator to alter the fluence of the radiation illuminating that skin portion, or take any other appropriate action, so as to lower the skin temperature to a safe range.

While a number of applications have been discussed above, the teachings of the invention are in no way limited to such applications, and the teachings of this invention may generally be employed for performing most applications where optical dermatology is now employed, or may in the future be employed. Because low power radiation sources are utilized, longer treatment times may be required. Such longer treatment times may be achieved by a single longer energizing of each radiation source, by successively pulsing a source to reduce power requirements on the power supply, or by pulsing the sources in a pattern which may involve returning to one or more (or all) of the sources multiple times during a single treatment. The later procedure may reduce thermal load on both the radiation sources and the subject's skin, reducing the need for cooling and enhancing safety. Treatments involving low-power, long duration irradiation are discussed, for example, in co-pending U.S. application Ser. No. 09/769,960 filed Jan. 25, 2001 the subject matter of which is, to the extent relevant, incorporated herein by reference.

Typical range of parameters of the apparatus proposed in present invention include wavelengths in the range of about 290-3000 nm, fluence is the range of about 0.5-1000 J/cm², and pulsewidth in the range of about 0.1 ms-1000 sec. More specific range of parameters has to be optimized for every particular treatment conditions for best safety and efficacy. Table 1 contains preferred wavelengths to be utilized in the treatment of specific dermatological conditions.

Relatively fail-safe and inexpensive methods and systems are provided for treating a wide range of medical and cosmetic dermatology problems, which are safe enough so as to permit their use outside of the usual institutional settings. For example, the apparatus of the present invention can be used in a beauty parlor, barber shop, spa, or even by the patient himself or herself in the home.

While the invention has been particularly shown and described above with reference to preferred embodiments, and variations thereof, it is to be understood that these embodiments have been presented for purposes of illustration only and that other applicators could be provided for treating other parts of the body and that other changes in form and detail can be made in the apparatus, the applicator thereof and the method for their use by one skilled in the art, while still remaining within the spirit and scope of the invention which is to be defined only by the appended claims.

Those skilled in the art will appreciate, or be able to ascertain using no more than routine experimentation, further features and advantages of the invention based on the above-described embodiments. Accordingly, the invention is not to be limited by what has been particularly shown and described, except as indicated by the appended claims. All publications and references are herein expressly incorporated by reference in their entirety.

What is claimed is:

1. An optical dermatology apparatus comprising:
a plurality of optical radiation sources;
a mount in which said sources are positioned at selected locations, said mount being adapted for positioning adjacent a treatment region of a subject's body; and
controls for operating said sources in an irradiation pattern.

2. The apparatus of claim 1, wherein the irradiation pattern formed by the controls comprises a spatial pattern.

3. The apparatus of claim 1, wherein the irradiation pattern formed by the controls is a temporal pattern.

4. The apparatus of claim 3, wherein said sources are operated in a selected sequence to form the temporal pattern.

5. The apparatus of claim 1, wherein said controls comprise a power supply and a switching element which connects said power supply to said sources in said irradiation pattern.

6. The apparatus of claim 1, wherein said mount further comprises a component which protects a selected portion of the subject's treatment region by preventing application of light from said sources to said selected portion.

7. The apparatus of claim 6, wherein said treatment region is the subject's face and wherein said selected portion is the subject's eyes.

8. The apparatus of claim 6, wherein the apparatus further comprises an interlock which operates in conjunction with said controls to disable operation of said sources unless said component is properly positioned to protect said selected portion.

9. The apparatus of claim 1, wherein said mount further comprises a component which permits said mount to be fitted to said treatment region with substantially uniform spacing between each of said sources and said treatment region.

10. The apparatus of claim 1, wherein said mount comprises an optically transparent component between said sources and said treatment region.

11. The apparatus of claim 10, wherein said transparent component comprises an optical filtering component.

12. The apparatus of claim 10, wherein said component is a protective component for the subject.

13. The apparatus of claim 1, wherein the apparatus further comprises a surface reflecting radiation from said sources to said treatment region.

14. The apparatus of claim 1, wherein the apparatus further comprises a reflecting surface for each of said sources.

15. The apparatus of claim 1, wherein said mount comprises a circuit board, said sources being mounted to said board.

16. The apparatus of claim 1, wherein said sources are U-shaped lamps.

17. The apparatus of claim 1, wherein said sources are solid state light emitters.

18. The apparatus of claim 17, including a lens array for directing light from said light emitters to said treatment region.

19. The apparatus of claim 1, wherein said mount is adapted to fit to all or part of a subject's face.

20. The apparatus of claim 1, wherein said treatment region is one of the subject's face, arm, thigh, leg, arm, hand, neck, hairline, underarms, crouch area, bikini line, buttocks, breast, and stomach, wherein said mount is adapted to be fitted to the treatment region.

21. The apparatus of claim 1, wherein the apparatus further comprises a diagnostic tool mounted in said mount.

22. The apparatus of claim 21, wherein said controls operate in response to said diagnostic tool to control operations of said sources.

23. The apparatus of claim 1, wherein the apparatus further comprises sensors for detecting proper positioning of said mount to said selected treatment region; and wherein said controls operate in response to said sensors to operate said sources only when said mount is properly positioned.

24. A method of performing optical dermatology by operating at least some of a plurality of optical radiation sources mounted adjacent a treatment region of a subject in an irradiation pattern.

25. The method of claim 24, wherein subsets of said sources are operated simultaneously.

26. The method of claim 24, wherein said sources are operated in a selected sequence.

27. The method of claim 24, including protecting a selected portion of the treatment region by preventing application of light from said sources to said selected portion.

28. The method of claim 27, wherein said treatment region is the subject's face and wherein said selected portion comprises the subject's eyes.

29. The method of claim 27, wherein said protecting step further comprises detecting that said selected portion is properly protected, and enabling operation of said sources in response to said detection.

30. The method of claim 24, wherein the method further comprises setting a mount for said sources to said treatment region so as to provide substantially uniform spacing between each said source and said treatment region.

31. The method of claim 24, wherein said treatment region is one of the subject's lower face, entire face, arm, thigh, leg, arm, hand, neck, hairline, underarms, back, crouch area, bikini line, buttocks, breast, or stomach.

32. The method of claim 24, wherein the method further comprises detecting a condition in the treatment region.

33. The method of claim 32, wherein the method further comprises operating said sources in response to said detecting.

34. The method of claim 24, wherein the method further comprises sensing the proper positioning of a mount for said radiation sources to said treatment position; and operating said sources only when said sensing step indicates that said mount is properly positioned.

35. An optical dermatology apparatus, comprising
a mount adapted for positioning in proximity of an area of a patient's skin,
one or more radiation sources disposed in said mount for irradiating at least a portion of said area of the patient's skin, and
a control circuitry electrically coupled to said radiation sources for actuating an irradiation pattern of said radiation sources for performing a treatment protocol.

36. The apparatus of claim 35, wherein said mount is shaped so as to substantially conform to a contour of a patient's body part.

37. The apparatus of claim 35, wherein said treatment protocol comprises selecting wavelengths appropriate for a dermatological condition.

38. The apparatus of claim 35, wherein the control circuitry can be programmed to selectively treat a portion of the treatment area.

39. The apparatus of claim 35, wherein the control circuitry controls activates the selected pattern of said radiation sources in a selected sequence.

40. The apparatus of claim 35, wherein said mount is disposable.

41. The apparatus of claim 35, wherein the apparatus further includes an optically transparent sheath adapted to couple to said mount, wherein said sheath conforms to the treatment area.

42. The apparatus of claim 41, wherein the sheath is user-replaceable.

43. An optical dermatology system, comprising an applicator, comprising
a mount for positioning in proximity of an area of a patient's skin,
a plurality of radiation sources disposed in said mount,
one or more sensors disposed in said mount for collecting any of diagnostics or monitoring data associated with at least a portion of said area of the patient's skin,
a control circuitry electrically coupled to said radiation sources for selective actuation thereof, and
a computer in communication with said applicator, said computer receiving data from said sensors and transmitting control signals to said control circuitry based on analysis of said data.

44. The optical dermatology system of claim 43, wherein said control signals cause actuation of at least selected ones of said radiation sources in a selected sequence.

45. The optical dermatology system of claim 43, wherein said computer communicates with said applicator via a wireless communications link.
